(19) United States
(12) Patent Application Publication
(10) Pub. No.: US 2002/0077595 A1
(43) Pub. Date: Jun. 20, 2002

(54) ENDOVASCULAR CORONARY SINUS CATHETER AND METHOD OF USE

(76) Inventors: RONALD R HUNDERTMARK, SAN MATEO, CA (US); CRAIG L ADAMS, SAN RAMON, CA (US)

Correspondence Address: Philip S Johnson, One Johnson & Johnson Plaza, New Brunswick, NJ, 08933-7003, US

(21) Appl. No.: 09/097,861

(22) Filed: Jun. 15, 1998

(51) Int. Cl. A61M 29/00

(52) U.S. Cl. 604/103.09; 604/96.01

(57) ABSTRACT

The coronary sinus catheter has a balloon for occluding the coronary sinus and a lumen for delivering fluid distal to the occluding member. The catheter has a proximal portion which is relatively stiff to provide column strength to facilitate insertion, advancement and steering of the catheter. The catheter also has a distal portion which is more flexible than the proximal portion to provide an atraumatic, kink-resistant distal end. The flexible, distal portion also helps prevent damage to the coronary sinus and facilitates guiding the catheter into the coronary sinus.

### CROSS-REFERENCE TO RELATED APPLICATION

This application is a continuation-in-part of Ser. No. 08/903,502, filed Jul. 30, 1997 which is hereby incorporated by reference.

### BACKGROUND OF THE INVENTION

The present invention relates to an endovascular coronary sinus catheter. A specific application of the endovascular coronary sinus catheter is for delivery of cardioplegic fluid to arrest a patient's heart and/or maintain the heart in an arrested state.

A system for arresting a patient's heart and maintaining circulation of oxygenated blood in the patient is disclosed in U.S. Pat. No. 5,584,803 which is hereby incorporated by reference. The coronary sinus catheter delivers cardioplegic fluid in a retrograde direction through the coronary sinus to arrest the patient's heart and/or maintain the patient's heart in an arrested state. The catheter passes through a peripheral vein, such as the internal jugular vein, so that direct access to the patient's heart is not required. An advantage of passing the catheter through a peripheral vein is that less invasive surgical procedures can be performed. Less invasive cardiac procedures are described in U.S. Pat. No. 5,452,733 and U.S. Patent Application Ser. No. 08/465,383, filed Jun. 5, 1995 by Sterman et al., which are hereby incorporated by reference.

Another coronary sinus catheter is disclosed in U.S. Pat. No. 5,558,644 which is also hereby incorporated by reference. The coronary sinus catheter of U.S. Pat. No. 5,558,644 is sized to permit endovascular placement of the catheter through a peripheral vein while providing a cardioplege lumen of sufficient size to deliver cardioplegic fluid at desirable flow rates and pressures.

It is an object of the present invention to provide an improved endovascular coronary sinus catheter.

### SUMMARY OF THE INVENTION

In accordance with the object of the present invention, an improved endovascular coronary sinus catheter is provided.

The endovascular coronary sinus catheter has a stiff proximal portion and a flexible distal portion. The stiff proximal portion provides column strength and facilitates insertion, advancement and steering of the catheter. The flexible distal portion provides an atraumatic, kink-resistant distal end. The flexible distal portion also helps prevent damage to the coronary sinus and facilitates guiding the catheter into the coronary sinus. In a preferred embodiment, the proximal portion has a modulus of elasticity of at least 40,000 psi while the distal portion has a modulus of elasticity of no more than 19,000 psi.

A stylet is preferably used to shape the distal portion to facilitate entry into the coronary sinus. A first contamination guard protects the stylet from contamination and a second contamination guard protects the catheter from contamination before introduction into the patient.

These and other aspects of the invention will become evident in the following description of the preferred embodiment.

### BRIEF DESCRIPTION OF THE DRAWINGS

FIG. 1 shows a coronary sinus catheter according to the present invention for delivering cardioplegic fluid to a patient's coronary sinus;

FIG. 2 shows the coronary sinus catheter of FIG. 1;

FIG. 3 is a cross-sectional view of a proximal portion of the coronary sinus catheter of FIG. 2 taken along line A-A;

FIG. 4 shows a distal end of the coronary sinus catheter;

FIG. 5 is a cross-sectional view of a distal portion of the coronary sinus catheter;

FIG. 6 is a longitudinal cross-sectional view of FIG. 5 taken along line B-B;

FIG. 7 is a cross-sectional view which illustrates an intermediate step in forming the distal portion of the coronary sinus catheter;

FIG. 8 is a longitudinal cross-sectional view of FIG. 7 taken along line C-C; and

FIG. 9 shows a method of joining together the proximal and distal portions.

### DESCRIPTION OF THE PREFERRED EMBODIMENT

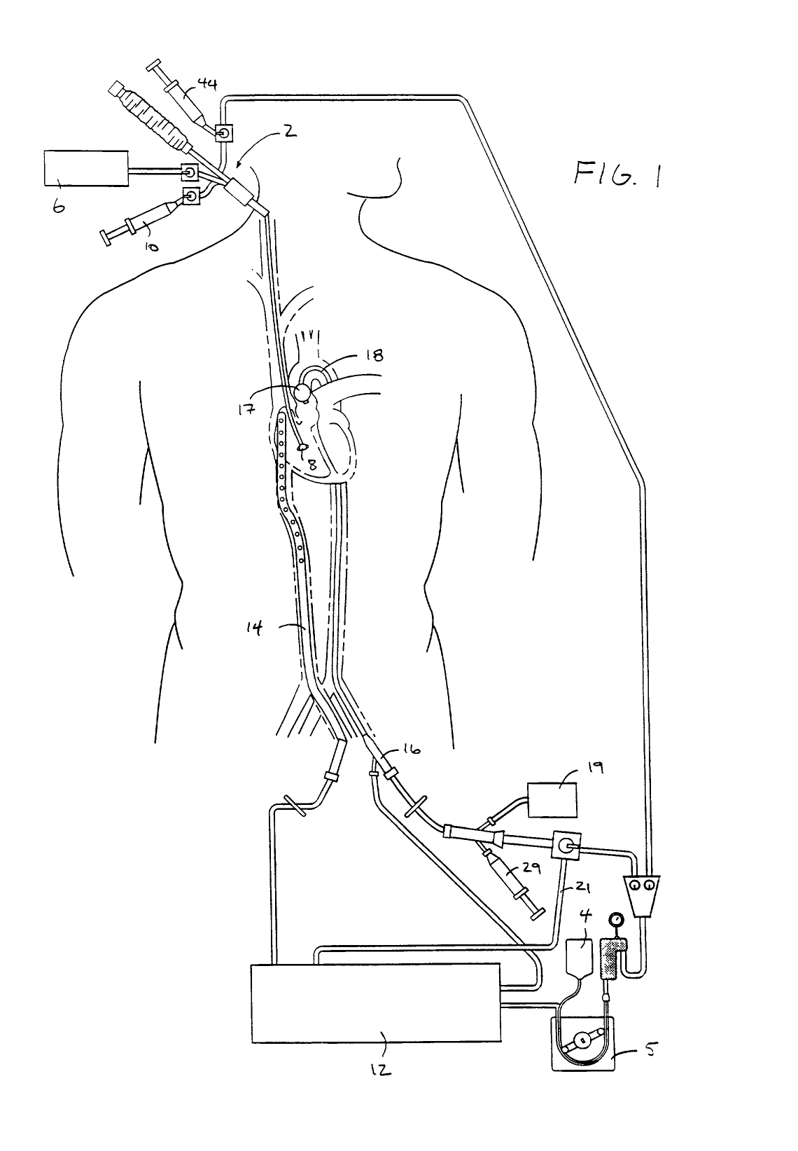

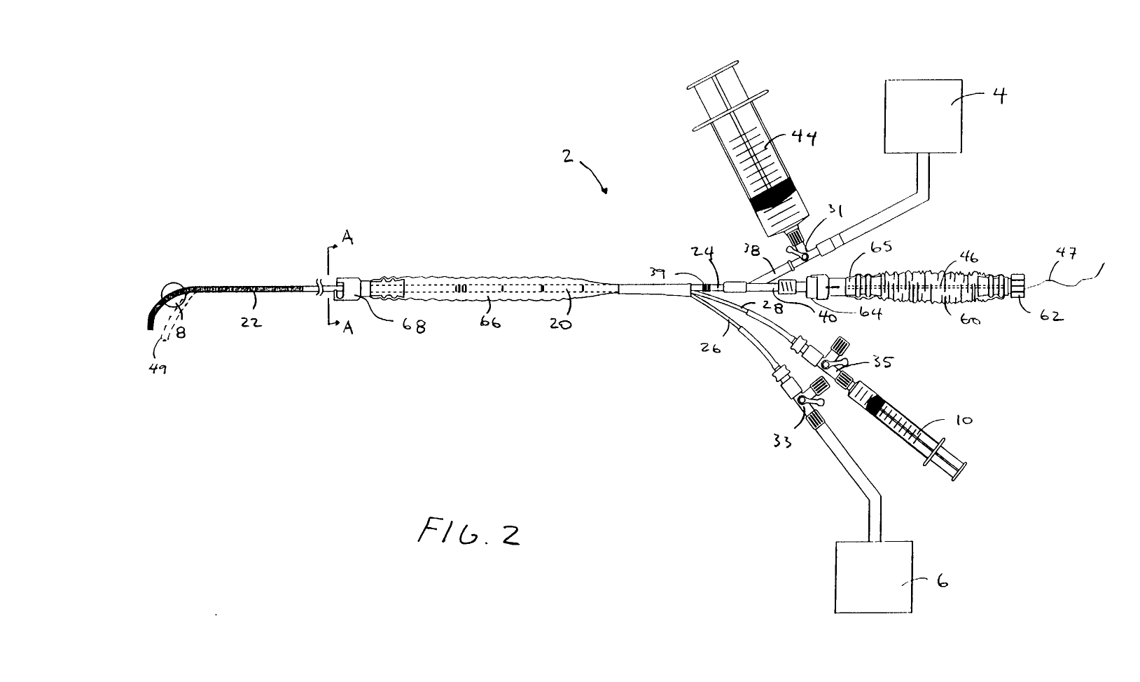

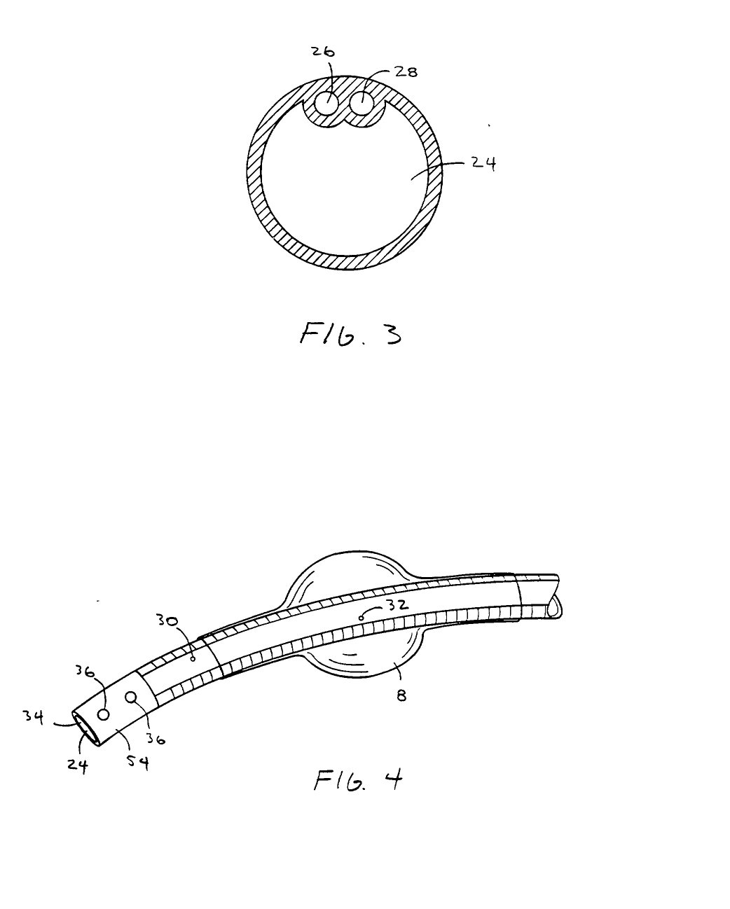

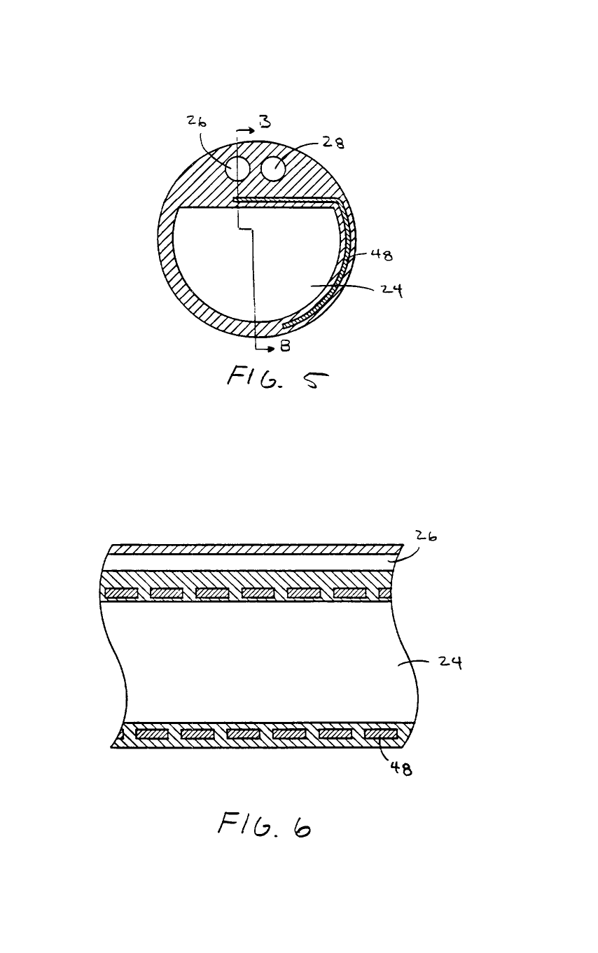

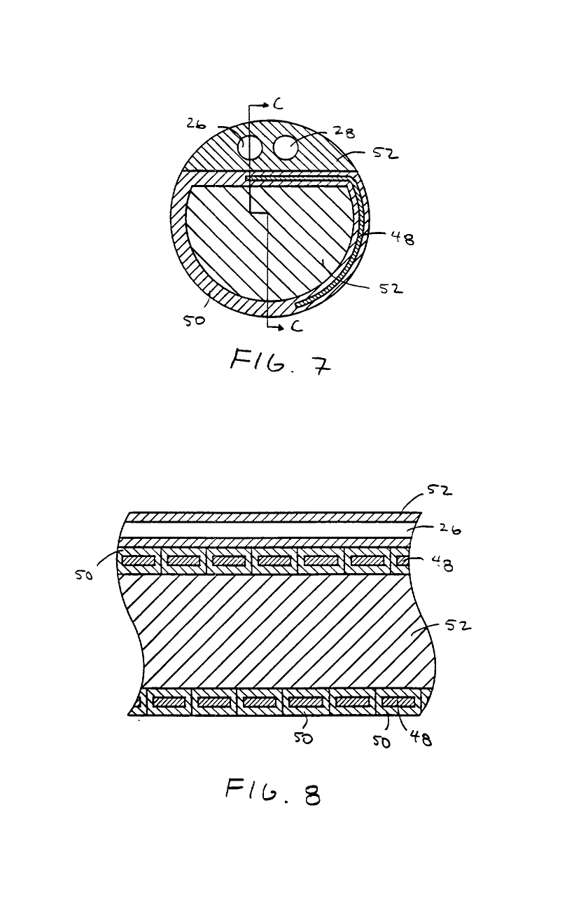

Referring to FIGS. 1 and 2, a coronary sinus catheter 2 in accordance with the present invention is shown. The coronary sinus catheter 2 is configured to be advanced through a peripheral vein, such as the internal jugular or left subclavian veins, and into the patient's coronary sinus.

In a specific application of the catheter 2 of the present invention, the catheter 2 is used to arrest and/or maintain a patient's heart in an arrested state by delivering cardioplegic fluid in a retrograde manner through the coronary sinus. A bypass system 12 maintains circulation of oxygenated blood in the patient when the patient's heart is arrested. The catheter 2 is coupled to a source of cardioplegic fluid 4 and a cardioplege pump 5 for delivering cardioplegic fluid to the patient's coronary sinus. The cardioplegic fluid is preferably mixed with oxygenated blood from the bypass system 12 to form blood cardioplege. An advantage of using blood cardioplegia is that oxygenated blood is also delivered to the patient's coronary vasculature. The catheter 2 is also coupled to a pressure sensor 6 for measuring pressure in the coronary sinus.

As mentioned above, the bypass system 12 maintains circulation of oxygenated blood in the patient when the patient's heart is arrested. Any bypass system 12 may be used with the coronary sinus catheter 2 of the present invention and one such system is described in U.S. Pat. No. 5,584,803 which is hereby incorporated by reference. The bypass system 12 preferably includes a pump, oxygenator, heat exchanger, and filter/bubble trap. A femoral venous cannula 14 withdraws blood from the patient and a femoral arterial cannula 16 returns oxygenated blood to the patient from the bypass system 12. Although it is preferred to pass the venous and arterial cannula through the femoral blood vessels, the venous and arterial cannulae 14, 16 may also be inserted directly into the vena cavae, right atrium and/or ascending aorta.

An aortic occlusion catheter 18 has a balloon 17 which occludes the ascending aorta. The aortic occlusion catheter 18 also delivers cardioplegic fluid, vents the ascending aorta and measures pressure in the ascending aorta. To this end, the aortic occlusion catheter 18 is coupled to the cardioplege pump 5, a vent line 21 and a pressure sensor 19. The aortic occlusion catheter 18 includes a lumen 13 having a valve 19 for controlling whether the lumen 13 is used for delivering cardioplegic fluid or venting of the aortic root through vent line 21. A syringe 29 filled with saline is used to inflate the balloon 17. The aortic occlusion catheter 18 preferably passes through the arterial cannula 16, however, the aortic occlusion catheter 18 may also be integrally formed with the arterial cannula 16 or completely separate from the arterial cannula 16. Although it is preferred to use the aortic occlusion catheter 18, a conventional cross-clamp may be used to occlude the ascending aorta and a conventional needle may be used for antegrade delivery of cardioplegic fluid and venting of the ascending aorta.

Referring to FIG. 2, the coronary sinus catheter 2 has a proximal portion 20 and a distal portion 22. The proximal portion 20 is relatively stiff to provide column strength and facilitate insertion, advancement and steering of the catheter 2. The distal portion 22 is more flexible than the proximal portion 20 to provide an atraumatic, kink-resistant distal end. The proximal portion 20 preferably has a modulus of elasticity of at least 35,000 psi, more preferably at least 40,000 psi and most preferably at least 45,000 psi. The distal portion 22 preferably has a modulus of elasticity of no more than 28,000 psi more preferably no more than 22.000 psi and most preferably no more than 16,000 psi. Alternatively, the proximal portion has a stiffness which is preferably at least 1.5, more preferably at least 2.1, and most preferably at least 2.7 times the stiffness of the distal portion. The modulus of elasticity of the proximal and distal portions 20, 22 is preferably determined empirically and a preferred method of testing is with an Olsen Stiffness Tester available from Tinius Olsen Testing Machine Co., Inc. of Willow Grove, Pa.

Referring to FIGS. 2 and 4, the catheter 2 also has an occluding member 8, preferably a balloon, which is used to occlude the coronary sinus so that the fluid delivered by the catheter 2 doesn't simply drain back into the right atrium. The occluding member 8 is made of polyurethane having a thickness of 0.0005 to 0.0007 inch. The occluding member 8 is inflated with a fluid from a source of inflation fluid 10. The source of inflation fluid 10, which is preferably a syringe, is preferably filled with contrast solution for fluoroscopic visualization. Alternatively, saline solution may be used if fluoroscopic visualization is unnecessary. Although it is preferred to use a balloon as the occluding member 8, a mechanically-actuated occluding member 8 may also be used.

Referring to FIGS. 2, 3 and 4, the catheter 2 has a lumen 24, a pressure lumen 26 and an inflation lumen 28 which are coupled to the source of cardioplegic fluid 4, pressure sensor 6 and source of inflation fluid 10, respectively. The pressure lumen 26 has an outlet 30 distal to the occluding member 8 for sensing pressure distal to the occluding member 8. The inflation lumen 28 has an outlet 32 for inflating the occluding member 8. The lumen 24 has an outlet 34 and side holes 36, preferably at least 0.025 inch in diameter, at the distal end to infuse fluid distal to the occluding member 8. Although it is preferred to provide the inflation lumen 28, the lumen 24 may also be used to inflate the occluding member 8 as is known in the art. The outer diameter of the catheter 2 is preferably no more than 0.125 inch and more preferably about 0.116 inch, the cross-sectional area of the inflation lumen 28 is preferably at least 0.000201 square inch and more preferably about 0.000254 square inch, the cross-sectional area of the pressure lumen 26 is preferably about 0.000254 square inch and the cross-sectional area of the lumen 24 is preferably about 0.00657 square inch.

The proximal portion 20 is preferably an extrusion made of polyether block amide having a durometer of 70D, however, any other suitable construction may be used. For example, the proximal portion 20 may include a reinforcing wire similar to the distal portion 22 as described below. As discussed above, the proximal portion 20 is stiff to provide column strength and facilitate advancement and steering of the catheter 2. The proximal portion 20 has a length of at least 30 cm. more preferably at least 40 cm and most preferably at least 50 cm.

The proximal portion 20 terminates at a trifurcation which splits the lumens 24, 26, 28 into independent lines having various connectors 25, 27, 29 and valves 31, 33, 35. The lumen 24 splits into first and second arms 38, 40 at a Y-connector 42 at the proximal end. The lumen 24 also has a bellows 39 before the Y-connector to prevent kinking. The first arm 38 is coupled to the source of cardioplegic fluid 4 and a source of contrast 44, which is preferably a syringe, for fluoroscopic visualization. A valve regulates flow through the lumen from the source of cardioplegic fluid 4 and source of contrast 44. In addition, a guidewire (not shown) may be inserted into the first arm 28 to facilitate placement of the catheter 2 into the coronary sinus.

The second arm 40 of the Y-connector 42 receives a stylet 46 which is used to shape the distal portion 22 of the catheter 2. The stylet 46 preferably has a shaped distal end which gives the distal end of the coronary sinus catheter 2 the curved shape of FIG. 2. The curved shape facilitates introducing the distal end into the coronary sinus when the catheter 2 extends through the peripheral vein. The stylet 46 may also be malleable so that it can be shaped by the user. The stylet 46 may also include a pull wire 47 for moving the distal end of the stylet 46 to the curved shape of line 49. Alternatively, the stylet 46 may be substantially straight in the unbiased condition with the pull wire 47 being used to move the distal portion 22 of the catheter 2 into the solid-line position of FIG. 2. Although it is preferred to use the stylet 46 to shape the catheter 2, the catheter 2 itself may be shaped so that the distal end is directed toward the coronary sinus when the catheter 2 extends through the peripheral vein.

Referring to FIGS. 5 and 6, cross-sectional views of the distal portion 22 are shown. The distal portion 22 is preferably reinforced with an elongate member 48, preferably a wire, to provide hoop strength and kink-resistance. As described above, the distal portion 22 is more flexible than the proximal portion 20 to provide an atraumatic, kink-resistant distal end. The elongate member 48 preferably passes around only the lumen 24, however, the elongate member 24 may also extend around the other lumens 26, 28. An advantage of winding the elongate member 48 around only the lumen 24 is that the outlets 32, 30 for the inflation and pressure lumens 28, 26 do not need to be cut through the elongate member 24. The distal portion 22 has a length of 2-5 cm, more preferably 2-4 cm and most preferably about 3-3.5 cm. Alternatively, the distal portion 22 is preferably at least 5 cm, more preferably at least 10 cm. and most preferably at least 15 cm.

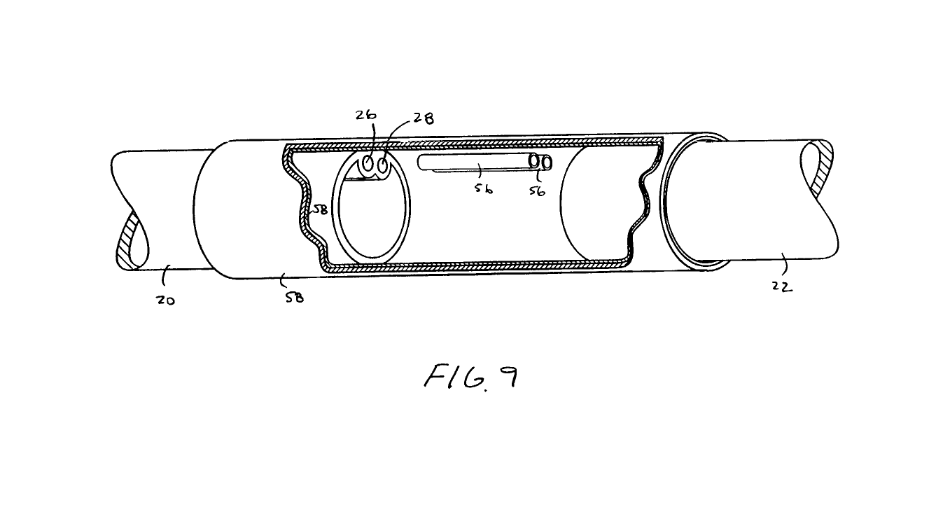

The method of forming the distal portion 22 is now described with reference to FIGS. 7 and 8. FIGS. 7 and 8 correspond to the cross-sectional view of FIGS. 5 and 6 in that FIGS. 7 and 8 show the distal portion 22 before heating and FIGS. 5 and 6 show the distal portion 22 after heating to form an integrated structure. The elongate member 48 is first coated with a coating 50, preferably by extrusion, and wrapped around a D-shaped mandrel 52 in a helical manner. The coating 50 may be any suitable polymer and is preferably polyether block amide having a durometer of 35D The elongate member 48 is preferably stainless steel ribbon having a thickness of 0.003 inch and a width of 0.012 inch After coating the elongate member 48 with the coating 50, the resulting coated elongate member 48 preferably has a width of 0.017 inch and a thickness of 0.009 inch Although it is preferred to use a rectangular cross-sectional shape, the elongate member 48 may also have a circular cross-sectional shape. A method of forming tubular structures in this manner is described in U.S. patent application Ser. Nos. 08/612,230 and 08/749,683 which are hereby incorporated by reference.

A two-lumen element 52, which forms the pressure and inflation lumens 26, 28, is then positioned against the coated, elongate member 48. Teflon-coated wire blockers (not shown) are inserted into the pressure and inflation lumens 26, 28 to prevent collapse during heating. A shrink tube (not shown) is then positioned around the entire structure and the entire structure is heated to form the integrated structures of FIGS. 5 and 6. The two-lumen element 52 is preferably made of a higher durometer polymer than the coating 50 so that the element 52 provides tensile strength and minimizes elongation. The softer coating 50 provides the catheter 2 with soft, pliable, atraumatic characteristics. The element 52 may be made of any suitable polymer and is preferably polyesther block amide having a durometer of 55 D.

Referring again to FIG. 4, the distal end of the catheter has a soft tip 54 to prevent injury to the patient when positioning and advancing the catheter 2. The tip 54 is preferably made of 25D polyether block amide having a thickness of 0.018 inch The tip 54 is heat bonded to the end of the distal portion 22 after forming the distal portion 22 in the manner described above.

Referring to FIG. 9, a partially exploded view illustrating a method of joining the proximal and distal portions 20, 22 together is shown. FIG. 9 shows the proximal and distal portions 20, 22 separated for the purpose of illustration, however, the proximal and distal portions 20, 22 are brought into contact with one another end-to-end when bonding them together. Tubes 56 are positioned in the pressure and inflation lumens 26, 28 bridging the proximal and distal portions 20, 22 to ensure that the lumens 26, 28 are sealed at the connection between the proximal and distal portions 20, 22. A pair of sheaths 58 are positioned over the intersection of the proximal and distal portions 20, 22 to ensure closure of the lumen 28. The tubes 56 are preferably made of polyimide having a thickness of 0.20 inch and each sheath 58 is preferably made of polyether block amide having a thickness of 0.002 inch. A heat shrink tube (not shown) is positioned over the sheaths 58 and blockers (not shown) are inserted into the lumens to 24, 26, 28 prevent the lumens from collapsing during heating. The entire structure is then heated to fuse the proximal and distal portions 20, 22 together. After heating, the proximal and distal portions 20, 22, tubes 56, and sheaths 58 form an integrated structure. Although it is preferred to use the tubes 56 and sheaths 58, the proximal and distal portions 20, 22 may be joined together in any other manner.

Referring again to FIG. 2, a first contamination guard 60 protects the stylet 46 against contamination before insertion into the patient. The proximal end of the stylet 46 and the first contamination guard 60 are mounted to a holder 62 which is manipulated to move the stylet 46. The distal end of the first contamination guard 60 engages a valve 64. The valve 64 has a seal (not shown) which prevents fluid from passing between the valve 64 and stylet 46. The distal end of the first contamination guard 60 has a valve 65 that locks onto the stylet 46 to ensure that the stylet 46 rotates with the catheter 2 when the catheter 2 is manipulated. Before delivering cardioplegic fluid, the stylet 46 is withdrawn until the distal end is within the second arm 40 and, therefore, does not impede delivery of cardioplegic fluid. The first contamination guard 60 is sized long enough so that the distal end of the stylet 46 can be withdrawn into the second arm 40. A second contamination guard 66 protects the catheter 2 against contamination before introduction into the patient. The second contamination guard 66 has a connector 68 which engages a conventional introducer sheath (not shown) which is used to introduce the catheter 2 into the patient. The first and second contamination guards 60, 66 are made of polyethylene having a thickness of about 0.002 inch. The catheter 2 shaft also has markers 68, preferably at 5 cm intervals beginning 10 cm from the distal tip, for use during placement of the catheter 2.

A method of using the coronary sinus catheter 2 is now described. The following describes a percutaneous method of inserting the catheter 2, however, any other method, such as surgical cut-down, may also be used. A needle (not shown) is inserted into the internal jugular vein and a guidewire (not shown) is fed through the needle. The needle is then removed leaving the guidewire in place. An introducer sheath having a dilator (not shown) is passed over the guidewire and into the vein. The dilator and wire are then removed leaving only the introducer sheath in the patient's vein.

The tip of the catheter 2 is then inserted into the introducer sheath and the connector 68 of the second contamination guard 66 is coupled to the introducer sheath. The coronary sinus catheter 2 is then advanced through the internal jugular vein, through the superior vena cava, and into the right atrium. Once the coronary sinus catheter 2 is in the right atrium, the stylet 46 directs the tip 54 toward the coronary sinus. If the tip 54 does not readily pass into the coronary sinus, a guidewire (not shown) is advanced beyond the distal end of the catheter 2. Once the guidewire is placed into the coronary sinus, the catheter 2 is advanced over the guidewire and into the coronary sinus. The guidewire is then removed and the stylet 46 is retracted until the distal end is withdrawn into the second arm 40 of the lumen 24. Proper positioning may be confirmed fluoroscopically by inflating the occluding member and delivering contrast through the lumen 24 or by monitoring the pressure lumen which indicates ventricular pressure when the coronary sinus is occluded. Positioning may also be verified via transesophageal echocardiographic imaging.

When it is desired to deliver the cardioplegic fluid, the occluding member 8 is inflated to occlude the coronary sinus. Cardioplegic fluid is then delivered through the lumen 24 from the source of cardioplegic fluid 4. Cardioplegic fluid may be delivered to arrest the patient's heart and/or to maintain the patient's heart in an arrested state. In order to maintain the heart in an arrested state, cardioplegic fluid is periodically infused into the coronary sinus.

While the above is a preferred description of the invention, various alternatives, modifications and equivalents may be used without departing from the scope of the invention. Therefore, the above description should not be taken as limiting the scope of the invention which is defined by the claims. For example, although the catheter 2 is described in connection with stopping a patient's heart and maintaining the heart in an arrested state, the catheter 2 may be used for endovascular access of the patient's coronary sinus for any other reason.

What is claimed is:

1. An endovascular coronary sinus catheter, comprising: a first lumen, an occluding member, a proximal portion, a distal portion, and a length sufficient to reach a patient's coronary sinus when the proximal portion extends through a peripheral vein, the first lumen having an outlet configured to infuse a fluid distal to the occluding member, the occluding member being sized and configured to occlude a patient's coronary sinus, the proximal portion having a modulus of elasticity of at least 30.000 psi and the distal portion having a modulus of elasticity of no more than 20,000 psi the proximal portion having a length of at least 30 cm and the distal portion having a length of 2-5 cm.

2. The catheter of claim 1, wherein: the proximal portion has a modulus of elasticity of at least 35,000 psi.

3. The catheter of claim 1, wherein: the distal portion has a modulus of elasticity of no more than 18,000 psi.

4. The catheter of claim 1, further comprising: a pressure lumen having an outlet positioned distal to the occluding member for measuring pressure distal to the occluding member.

5. The catheter of claim 1, wherein: the distal portion includes a reinforcing wire wrapped in a helical manner.

6. The catheter of claim 1, wherein: the distal portion includes a reinforcing wire and the proximal portion is an extrusion.

7. The catheter of claim 1, further comprising: a stylet slidably disposed within the lumen, the stylet having a shaped end which is configured to facilitate positioning a distal end of the coronary sinus catheter into the patient's coronary sinus when the proximal portion extends through a peripheral vein.

8. The catheter of claim 1, wherein: the lumen has a bellows connector and a Y-connector which splits the lumen into first and second arms.

9. The catheter of claim 8, further comprising: a stylet slidably received in the second arm.

10. The catheter of claim 9, further comprising: a contamination guard configured to surround the stylet, the contamination guard and stylet being mounted to a holder for manipulating the stylet, the contamination guard having a length sufficient to withdraw a distal end of the stylet into the second arm.

11. The catheter of claim 1, further comprising: a contamination guard extendable over the proximal portion and the distal portion, the contamination guard having a connector configured to engage an introducer sheath.

12. A method of accessing a patient's coronary sinus, comprising the steps of:
providing a coronary sinus catheter having a proximal portion, a distal portion, and a distal end, the proximal portion having a modulus of elasticity of at least 30,000 psi and a length of at least 30 cm the distal portion having a modulus of elasticity of no more than 20,000 psi and a length of 2-5 cm;
creating an opening in a peripheral vein of a patient;
inserting the coronary sinus catheter through the opening in the peripheral vein of the patient;
advancing the coronary sinus catheter through the peripheral vein and into the right atrium;
positioning the distal end into the patient's coronary sinus.

13. The method of claim 12, wherein:
the providing step is carried out with the coronary sinus catheter including a lumen having an outlet.

14. The method of claim 13, further comprising the step of:
coupling the lumen to a source of cardioplegic fluid; and
infusing cardioplegic fluid into the patient's coronary sinus.

15. The method of claim 14, further comprising the step of:
occluding the patient's ascending aorta.

16. A method of joining a first tubular body to a second tubular body, comprising the steps of:
providing a first tubular body and a second tubular body, the first and second tubular bodies each having a first lumen and a second lumen;
positioning an end of the first tubular body adjacent an end of the second tubular body;
positioning a tube in the first lumen of thc first and second tubular members, the tube extending through the first and second tubular bodies; and
heating the first and second tubular bodies thereby bonding the first and second tubular bodies together.

17. The method of claim 16, further comprising the step of:
positioning a sheath around an outer surface of the first and second tubular members, the sheath extending around the ends of the first and second tubular bodies;
the heating step being carried out so that the sheath and first and second tubular bodies melt and fuse together.
